(19) United States
(12) Patent Application Publication
(10) Pub. No.: US 2008/0252851 A1
(43) Pub. Date: Oct. 16, 2008

(54) Binocular Indirect Ophthalmoscope Generating Erect Ocular Fundus Image

(76) Inventor: Tarek Abd-Elkarem Mohammed Shazly, Assuit (EG)

Correspondence Address: SWANSON & BRATSCHUN, L.L.C., 8210 SOUTHPARK TERRACE, LITTLETON, CO, 80120, US

(21) Appl. No.: 12/088,398

(22) PCT Filed: Sep. 20, 2006
(86) PCT No.: PCT/EG2006/000034

§ 371 Date: Jul. 2, 2008

(30) Foreign Application Priority Data
Oct. 2, 2005 (EG) 2005100432

(51) Int. Cl. A61B 3/12

(52) U.S. Cl. 351/221; 351/205

(57) ABSTRACT

This invention relates to equipment indirectly visualizing the fundus of the eye through a condensing lens. It is characterized with light weight and relatively of simple structure, contrary to the common indirect opthalmoscopes, whereby the image of the back of the eye can be seen upright. Thus, many complications presented during the training on the equipment application can be avoided averted as the image obtained needs no interpretation and any pathological change can be determined by the doctor in its exact location.

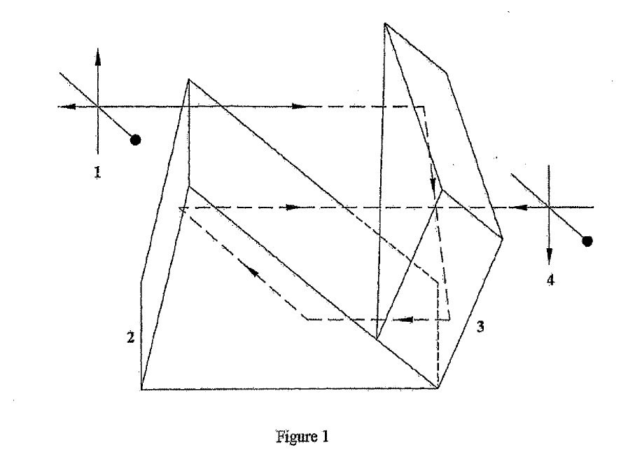

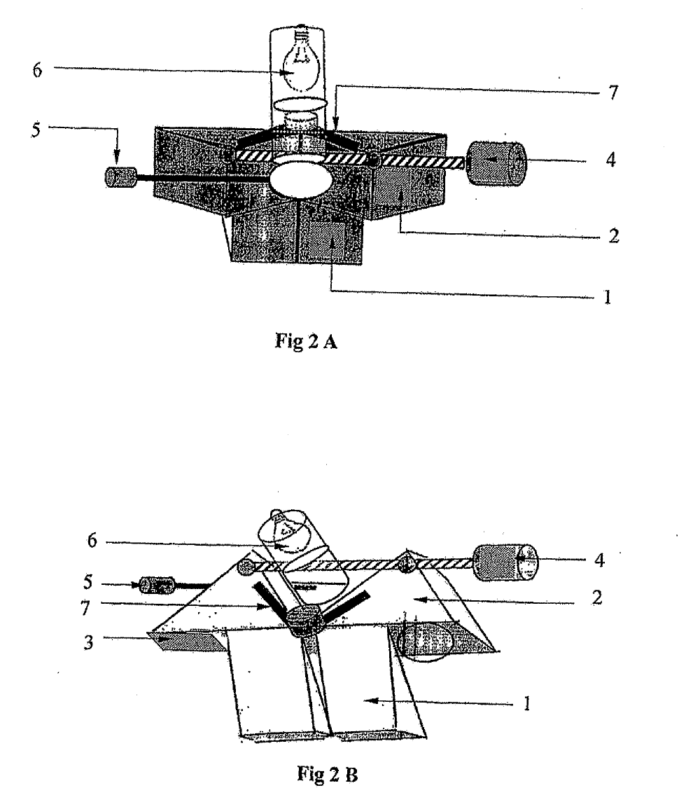

### TECHNICAL FIELD

This device has to do with group of devices commonly known as Binocular indirect opthalmoscopes that medical devices used to examine the back of the eye by an ophthalmologist or optometrist for diagnosis of different conditions affecting the back of the eye.

### BACKGROUND ART

The opthalmoscope for indirectly imaging Back of the eye is one of the most essential equipments regularly used by the ophthalmologist in examining the patients. It is indispensable in observing retina, optic nerve and the vitreous body. Not withstanding the various models and types labeled as equipments indirectly examining the fundus of the eye, yet, all of which are based on the same optic basis, that is, the usage of condensing lens (high powered convex lens) deposited before the patient eye through which the doctor can see an inverted real image of the Back of the eye where the vision system before the doctor eye converts the said inverted image into two inverted images before the doctor's two eyes respectively forming a stereoscopic and inverted image of the eye ground.

The current equipments available for such purpose are of a defect, being the formation of inverted image of the Back of the eye, which, in turn, causes a great con fusion for the beginners of ophthalmologists upon applying the said opthalmoscope. As they have to become familiar with the inverted image of the Back of the eye contrary to the majority of the diagnostic equipments used in the ophthalmic medicine and surgery. It thus enlarges the training period on the said equipment and makes it more complicated in application. Such conventional equipment is further expensive and usually of heavier weight.

This invention provides an image having the same merits of the conventional equipments as to the extension and clarity of the vision field, however, it is superseded by the formation of upright image. Thus it is not required to conceive the resulted image in reversal position by the doctor. The said equipment is of low costs and to some extent of simple structure, characterizing with the following:
(1) The applied technique in obtaining upright image allows the reduction of total size for the vision unit plus its light weight being much easier in application. The said equipment can also be provided with additional parts and accessories for more prospective functions compatible with the components of the conventional equipments as condensing lenses, learning mirrors and the like.
(2) With respect to the simplicity of the design, it is not based on a bij number of optic medias or inversive planes, so, the lucidity of the image and being feasible to the practicality and applicability.
(3) Technically, the optic basis utilized in the relevant invention allows to produce a stereoscopic image while extending the visior field.
(4) It is of low costs to great extent, thus, reducing the total expenses of manufacturing.

### DISCLOSURE OF INVENTION

The Binocular Indirect Opthalmoscope Generating Erect Ocular Fundus Image works through inverting the already inverted image taken for the back of the eye by using a condensing lens acting vertically and horizontally to obtain an upright image. This is achieved by using a number of inversive planes arranged into two arrays; one of which is a primary vertically inversive array inverting the image to the perpendicular, and the other is a secondary horizontally inversive array inverting the image horizontally from one side to another as well as displacing it laterally in consistence with the distance between the two pupils of the user. In such way, the inverted image is converted by the condensing lens into two spaced upright images before each eye after crossing a convex lens of 2 diopter. The said equipment is also provided with a light source and a head belt and the other mechanical parts and binders necessary for an indirect opthalmoscope.

The Inverted—reversed fundus image formed by the condensing lens is received by the first (central) reflecting surfaces either mirrors, Porro type 1 or type 2 prisms.

In case that Porro type 1 prisms or equivalent mirror arrangement is used the image formed by the condensing lens will be received by the vertical prisms/mirrors and be splitted into 2 slightly dissimilar images that will be also vertically reversed and will be received by the horizontal prisms/mirrors that will invert the 2 images laterally to obtain 2 final images that will be re-inverted and re-reversed and laterally shifted to be suitable for the examiners interpupillary distance.

In the Porro prism type 2 or equivalent mirror arrangement model, the image formed by the condensing lens will be received by the central prisms/mirrors to be rotated both vertically and horizontally and be delivered to the lateral prisms/mirrors to be completely re-inverted, re-reversed and laterally shifted to fit the examiners interpupillary distance.

The device contains also means for interpupillary distance control e.g tilting the prisms/mirrors as in Porro type 1 prisms or equivalent mirror model or horizontally separating them as in Porro 2 or equivalent mirror model.

The said equipment is labeled as a type of opthalmoscopes examining the Back of the eye in indirect way. It can generally be used in diagnosing all diseases of retina and the vitreous body, and in running the common retinal operations. It has an additional advantage of scoping the eye in an upright image.

### BRIEF DESCRIPTION OF DRAWINGS

FIG. (1)
1—An inverted image if the Back of the eye
2—A horizontal prism of right angle
3—A vertical prism of right angle
4—The final upright image of the Back of the eye

FIG. (2)
1—First group of reflecting surfaces,
2—Second group of reflecting surfaces,
3—Plus lens (+2) inside the ocular piece,
4—Light beam controller
5—Interpupillary distance controller,
6—Source of light,
7—Mechanical parts

1. Binocular Indirect opthalmoscope used for scoping an upright image for the fundus of the eye works on inverting already inverted image taken for the back of the eye via a condensing lens acting vertically and horizontally to obtain an upright image. This is achieved by using a number of inversive planes arranged into two groups; to reverse the position of the inverted image (turning the upper to lower), and inverting the image horizontally from one side to another to produce an upright image.

2. Binocular Indirect opthalmoscope according to claim 1, wherein the inversive planes comprise a pair of double Porro prisms either type 1 or 2 or equivalent mirror arrangement.

3. Binocular Indirect opthalmoscope according to claim 1, wherein the two prisms of right angles or equivalent mirror arrangement inverting the image vertically are placed in adjacent.

4. Binocular Indirect opthalmoscope according to claim 1, wherein the two prisms of right angles or equivalent mirror arrangement inverting the image horizontally are placed in adjacent and perpendicular to the two vertical prisms equivalent mirror arrangement, and further function on laterally displacing the two image both the right and the left in consistence with the distance between the two pupils of the user.

5. Binocular Indirect opthalmoscope according to claim 1 encompasses a right eyepiece and a left eyepiece.

6. Binocular Indirect opthalmoscope according to claim 1, wherein each eyepiece contains a lens.

7. Binocular Indirect opthalmoscope according to claim 6, wherein the lens in each eyepiece is of +2 diopter.

8. Binocular Indirect opthalmoscope according to claim 1, wherein the two eyepieces are movable in consistence with the distance between the two pupils of the user.

9. Binocular Indirect opthalmoscope according to claim 5, wherein the primary and secondary inversive arrays and the two eyepieces are incorporated in the viewing unit which also includes the cover connected to the head belt for supporting the viewing unit on the head of the user.

10. Binocular Indirect opthalmoscope according to claim 9 includes a lighting unit connected to the viewing unit providing the source for lightening the fundus of the eye examined. It may be connected to external power source or a battery chargeable allowing the movability of the equipment.

11. Binocular Indirect opthalmoscope according to claim 10 includes supplementary elements directing the light out of the light source and the size of the light spot falling on the eye examined.
